(19) United States
(12) Patent Application Publication
(10) Pub. No.: US 2023/0240952 A1
(43) Pub. Date: Aug. 3, 2023

(54) USE, IN AQUEOUS-ALCOHOLIC COMPOSITIONS, OF A COPOLYMER OBTAINED BY PRECIPITATION POLYMERIZATION

(71) Applicant: SNF Group, Andrezieux-Boutheon (FR)

(72) Inventors: Frédéric BLONDEL, (US); Dimitri MATIOSZEK, (US)

(73) Assignee: SNF Group, Andrezieux-Boutheon (FR)

(21) Appl. No.: 18/044,015

(22) PCT Filed: Sep. 6, 2021
(86) PCT No.: PCT/FR2021/051521

§ 371 Date: Mar. 3, 2023

(30) Foreign Application Priority Data
Sep. 7, 2020 (FR) 2009044

(51) Int. Cl. A61K 8/34; C08F 220/58; C08F 220/06; A61K 8/81; A61Q 17/00

(52) U.S. Cl. CPC A61K 8/34; C08F 220/585; C08F 220/06; A61K 8/345; A61K 8/8147; A61K 8/8158; A61Q 17/005; C08F 2810/20; C08F 2800/10; A61K 2800/48

(57) ABSTRACT

This invention relates to the use, for the manufacture of a hydroalcoholic composition comprising at least water and at least one alcohol of a branched or cross-linked copolymer obtained from:
(i) 20 mol % to 94.99 mol % of at least one monomer containing an acrylic group and bearing at least one weak acid function,
(ii) 5 mol % to 79.99 mol % of at least one monomer bearing at least one strong acid function,
(iii) 0.01 mol % to 4 mol % of a cross-linking/branching agent,
said copolymer being obtained by precipitation polymerization, in a solvent comprising at least one alcohol.

### FIELD OF THE INVENTION

This invention relates to the field of hydroalcoholic compositions. More precisely, the invention relates to the use of a copolymer obtained by precipitation polymerization in hydroalcoholic compositions.

### PRIOR ART

Viruses are the source of some of the most common human illnesses, including the common cold, chicken pox, and herpes. Certain serious diseases, including those responsible for many of the epidemics of modern society, such as Ebola, AIDS, SARS, and SARS-CoV-2, are also caused by viruses.

Viruses are infectious agents consisting of a nucleic acid (DNA/RNA) within a protein coating that attaches to the host cells of living organisms and replicates by forcing the host cell to copy the virus' genetic material. Because viruses can replicate in the body despite host defense mechanisms, they can cause lifelong chronic infections. In addition to their devastating ability to cause serious illness, viruses are also easily transmitted through bodily fluids, contaminated food/drink, insects, and physical contact.

High alcohol compositions generally help prevent the transmission of viruses and other pathogens through the skin because they provide virucidal efficacy without skin irritation. These hydroalcoholic compositions kill a wide range of viruses and other pathogens that may be present on the skin, especially the hands.

Thickening polymers are commonly added to hydroalcoholic compositions to give them rheological properties and thus make their application efficient and practical.

Different types of polymers formed from at least one monomer containing a weak acid function are generally used as thickening and/or stabilizing agents in different types of applications. For example, we may cite patents FR 2 810 545 and FR 2 873 126 or U.S. Pat. No. 3,724,547.

Patent EP 1 047 716 describes polymers based on acrylic acid and 2-acrylamido methylpropane sulfonic acid obtained by inverse emulsion polymerization. Although these polymers appear to have a thickening effect in aqueous media, they are not very effective in hydroalcoholic compositions. Moreover, they are not able to provide a transparent hydroalcoholic gel.

Patent JPH 09157130 describes polymers based on acrylic acid and 2-acrylamido-2-methylpropane sulfonic acid obtained by different polymerization techniques. The exemplified polymers are obtained by dispersion polymerization.

Although the polymers of the prior art are effective in aqueous solution, they do not allow interesting rheological properties in the presence of alcohol and a transparent hydroalcoholic gel.

### DISCLOSURE OF THE INVENTION

This invention relates to the use of a branched or cross-linked copolymer for the manufacture of a hydroalcoholic composition comprising at least water and at least one alcohol. Said branched or cross-linked copolymer is obtained from:

(i) 20 mol % to 94.99 mol % of at least one monomer containing an acrylic group and bearing at least one weak acid function,

(ii) 5 mol % to 79.99 mol % of at least one monomer bearing at least one strong acid function,

(iii) 0.01 mol % to 4.0 mol % of a cross-linking/branching agent, said copolymer being obtained by precipitation polymerization in a solvent comprising at least one alcohol.

The invention also relates to the use of this branched or cross-linked copolymer to thicken a hydroalcoholic composition comprising at least water and at least one alcohol.

The invention also relates to hydroalcoholic compositions comprising water, at least one alcohol, and this branched or cross-linked copolymer.

The phrase “monomer bearing at least one acid function” is meant to be understood as a monomer bearing one or more free or neutralized (i.e., salified by the action of a base) acid functions. When a monomer has more than one acidic function, it is possible to have only some of the acidic functions in neutralized form. The acid function(s) present may be a weak or strong acid function. In general, the monomers used in the invention only have weak acid functions or strong acid functions, and more preferably, monomers bearing only one acid function will be used.

All mole percentages are expressed relative to the total number of moles of monomers, including the cross-linking/branching agent. Of course, the skilled person will know how to adjust the different percentages to reach 100.

The copolymer is obtained by precipitation polymerization of at least one monomer comprising an acrylic group and bearing at least one weak acid function and at least one monomer comprising at least one strong acid function.

According to the invention, the monomer containing an acrylic group and bearing at least one weak acid function is advantageously chosen from acrylic acid, methacrylic acid, itaconic acid, crotonic acid, fumaric acid, maleic acid, and mixtures thereof. This monomer may be in acidic, partially salified, or fully salified form. The salified form preferably corresponds to an alkali metal salt, an alkaline earth metal salt, or an ammonium salt (NH₄⁺, primary, secondary, tertiary, or quaternary ammonium). Preferably, the monomer with a weak acid function is acrylic acid.

The copolymer preferably comprises between 20 and 89.99 mol % of at least one monomer containing an acrylic group and bearing at least one weak acid, more preferably between 30 and 89.99 mol %.

Preferably 10 to 100 mol % of the weak acid functions are salified, preferably 20 to 80 mol %, more preferably 30 to 70 mol %.

The monomer bearing at least one strong acid function is advantageously chosen from monomers bearing a sulfonic acid function such as acrylamido alkyl sulfonic acids, for example, 2-acrylamido-2-methylpropane sulfonic acid, or a phosphonic acid function. This monomer may be in acidic, partially salified, or fully salified form. The salified form preferably corresponds to an alkali metal salt, an alkaline earth metal salt, or an ammonium salt (NH₄⁺, primary, secondary, tertiary, or quaternary ammonium). Preferably, the monomer derived from a sulfonic acid is 2-acrylamido-2-methylpropane sulfonic acid.

According to the invention, the copolymer preferably comprises between 10 and 79.99 mol % of at least one monomer bearing at least one strong acid, more preferably between 10 and 69.99 mol % relative to the total amount of monomers making up the copolymer.

Preferably, 20 to 100 mol % of the strong acid functions are salified, more preferably 40 to 100 mol % and even more preferably 60 to 100 mol %.

In their salified form, the acid functions are in anionic form with a counterion or cation depending on the base used for neutralization, for example, of the Na⁺ type when sodium carbonate is used or NH₄⁺ when ammonia (gas) is used. Traditionally, control of the number of acid functions in salified form is ensured by the choice of the pH of the monomer solution, which will be adjusted according to the pKa of the acid functions present. Preferably, it is an NH₄⁺ salt.

Preferably, the acid functions are salified by a salifying agent selected from ammonia, sodium carbonate, potassium carbonate, ammonium carbonate, sodium bicarbonate, potassium bicarbonate, ammonium bicarbonate, and mixtures thereof. Preferably, the salifying agent is ammonia.

Optionally, the copolymer may comprise at least one nonionic monomer. The nonionic monomer or monomers that may be used in the context of the invention may be selected, in particular, from the group comprising water-soluble vinyl monomers. Preferred monomers belonging to this class are, for example, acrylamide, methacrylamide, N-isopropylacrylamide, N,N-dimethylacrylamide, and N-methylolacrylamide. Also, N-vinylformamide, N-vinyl acetamide, N-vinylpyridine, and N-vinylpyrrolidone, acryloyl morpholine (ACMO), and diacetone acrylamide may be used. One preferred nonionic monomer is acrylamide.

Optionally, the copolymer may comprise at least one cationic monomer. The cationic monomer or monomers that may be used in the context of the invention may be chosen, in particular, from monomers of the vinyl type possessing a quaternary ammonium function, in particular acrylamide, acrylic, allylic, or maleic monomers possessing a quaternary ammonium function. We may specifically mention, without limitation, quaternized dimethylaminoethyl acrylate (ADAME), quaternized dimethylaminoethyl methacrylate (MADAME), dim ethyl diallylammonium chloride (DADMAC), acrylamido propyltrimethyl ammonium chloride (APTAC), and methacrylamido propyltrimethyl ammonium chloride (MAPTAC).

Preferably, the copolymer comprises (excluding the cross-linking/branching agent) three monomers (terpolymer) or less, more preferably two monomers.

Preferably, the copolymer consists of the following monomers (excluding the cross-linking/branching agent): at least one monomer having an acrylic group and bearing at least one weak acid function and at least one monomer bearing at least one strong acid function, said monomers being as described above.

More preferably, the copolymer consists of the following monomers (excluding the cross-linking/branching agent): at least one acrylic acid monomer and at least one 2-acrylamido-2-methylpropane sulfonic acid (ATBS) monomer.

The cross-linking/branching agent is advantageously chosen from polyfunctional agents such as methylenebisacrylamide (MBA), ethylene glycol di-acrylate, polyethylene glycol dimethacrylate, diacrylamide, cyanomethylacrylate, vinyloxyethylacrylate vinyloxymethacrylate, triallylamine, trimethylolpropane triacrylate (TMPTA), tetraallylammonium chloride (TAAC), formaldehyde, glyoxal, glycidyl ethers such as ethylene glycol diglycidyl ether, epoxies, and mixtures thereof. Preferably, the cross-linking/branching agent is trimethylolpropane triacrylate (TMPTA).

According to the invention, the copolymer preferably comprises between 0.01 and 2.0 mol % of a cross-linking agent, preferably between 0.01 and 1.8 mol %.

The polymerization may be conducted with a transfer agent, for example, selected from methanol, isopropyl alcohol, sodium hypophosphite, 2-mercaptoethanol, sodium methallysulfonate, and their mixture. We may also mention transfer agents of the dithiocarbonate, dithiocarbamate, and trithiocarbonate type and mixtures thereof. Preferably, the amount of transfer agent is between 0 and 5000 ppm by weight with respect to the total weight of monomers (including the cross-linking/branching agent), and preferably between 10 and 2500 ppm.

Precipitation polymerization in a solvent medium consists of the polymerization of soluble monomers in a solvent, while the copolymer obtained is insoluble and thus precipitates into the solvent. The feasibility of this type of polymerization can be optionally ensured by the presence of particular surfactants giving adapted fluidity to the dispersion medium.

The solvent used for precipitation polymerization is preferably an alcohol comprising 1 to 4 carbons. It is advantageously selected from methanol, ethanol, propan-1-ol, isopropanol, tert-butanol, and mixtures thereof. Preferably, the solvent used is tert-butanol.

According to a particular embodiment, the polymerization may be performed in the presence of water. The solvent may comprise up to 10% by weight of water, preferably up to 5% by weight of water. In this case, the solvent consists of 90% to 100% by weight of an alcohol and 0% to 10% by weight of water.

Conventionally, the polymerization reaction is initiated by introducing a free radical initiator. As an example of a free radical initiator, we may mention the oxidizing-reducing couples with, among the oxidizing agents, cumene hydroperoxide or tertiary butylhydroxyperoxide, and among the reducing agents, persulfates such as sodium metabisulfite and Mohr's salt. Azo compounds such as 2,2′-azobis(isobutyronitrile), 2,2′-azobis(2-methylbutyronitrile), and 2,2′-azobis(2-amidinopropane) hydrochloride may also be used as well as peroxide compounds such as benzoyl peroxide, tert-butyl hydroperoxide, or lauroyl peroxide.

Preferably the active material represents 5% to 25% by weight of the reaction medium, more preferably 10% to 20%. The active material includes the monomers and the cross-linking/branching agent.

Once the polymerization is complete, the copolymer appears in the reaction medium as a precipitate. It is usually a white precipitate. It can be easily isolated using the usual separation (e.g., filtration or evaporation) and drying processes. The solvent can be removed by filtration or distillation.

Another aspect of the invention relates to hydroalcoholic compositions comprising:
a) 55% to 79.95% by weight of at least one alcohol A1,
b) 0.05% to 5% by weight of at least one branched or cross-linked copolymer as described above,
c) 15% to 44.95% by weight of water, preferably 20% to 44.95%,
d) 0% to 10% by weight of at least one additive.

According to the invention, alcohol A1 included in the hydroalcoholic solution is advantageously chosen from methanol, ethanol, isopropanol, butanol, pentanol, hexanol, their isomers, and mixtures thereof. Preferably, the alcohol is selected from ethanol and/or isopropanol.

According to the invention, the hydroalcoholic composition is advantageously transparent. Thus, the hydroalcoholic composition has a transmittance advantageously higher than 80%, preferably higher than 85%, and more preferably higher than 90%.

The transmittance values can be measured with a UV-photometer DR 3900 (HACH). Transmittance is the percentage of light transmitted through a 1 cm thick sample in a quartz cell at a wavelength between 400 nm and 800 nm. It is advantageously measured in the absence of additives, i.e., for a composition consisting of: a) 55 to 79.95% by weight of at least one alcohol A1, b) 0.05 to 5% by weight of at least one branched or cross-linked copolymer as described above, and c) 15 to 44.95% by weight of water.

According to the invention, the hydroalcoholic composition may comprise at least one additive (0-10% by weight of the composition). The additive is advantageously selected from surfactants, pH adjusting agents, fragrances, colorants, preservatives, and moisturizing agents.

Preferably, the hydroalcoholic composition comprises at least one moisturizing agent. The moisturizing agent may be selected from allantoin; pyrrolidonecarboxylic acid and salts thereof; hyaluronic acid and salts thereof; sorbic acid and salts thereof; amino acids, e.g., urea, lysine, arginine, cysteine, or guanidine; polyhydroxyalcohols such as glycerine, propylene glycol, hexylene glycol, hexanetriol, ethoxydiglycol, dimethiconecopolyol, and sorbitol, as well as esters thereof; polyethylene glycol; glycolic acid and glycolate salts (e.g., ammonium and alkyl quaternary ammonium); chitosan; aloe vera extracts; algae extracts; honey and extracts thereof; inositol; lactic acid and lactate salts (e.g., ammonium and alkyl quaternary ammonium); D-panthenol; magnesium ascorbyl phosphate; kojic acid; lactamide-monoethandamine; acetamide-monoethanolamine; their analogs, and mixtures thereof. Preferably, the moisturizing agent is glycerin.

The hydroalcoholic composition preferably comprises:
a) 60% to 69.9% by weight of at least one alcohol,
b) 0.1% to 3% by weight of at least one branched or cross-linked copolymer as described above,
c) 25% to 39.9% by weight of water,
d) 0% to 10% by weight of at least one additive.

The hydroalcoholic composition preferably comprises between 0.1% and 10% by weight of at least one moisturizing agent, preferably from 0.5% to 8% by weight, and more preferably, from 1% to 5% by weight, based on the total weight of the composition.

The invention and the advantages thereof will be better understood from the following examples given to illustrate the invention and not in a limiting manner.

### EXAMPLES OF EMBODIMENTS OF THE INVENTION

### Protocol for the Preparation of an ATBS/AA Copolymer by Precipitation Polymerization

460 g of tert-butanol comprising 2.5% by weight of water is introduced into a jacketed reactor equipped with a stirring paddle. 2-acrylamido-2-methylpropane sulfonic acid (ATBS) and acrylic acid (AA) are added with stirring. The medium is bubbled with ammonia gas for 30 minutes while checking that the pH does not exceed 7. The preparation is degassed in the reactor by injecting nitrogen for 30 minutes. During this time, the medium is gradually heated to 55° C. Trimethylolpropane triacrylate (TMPTA, cross-linking/branching agent) is then added, and the temperature of the medium is brought to 70° C. using a thermostatic bath. The polymerization reaction is initiated by adding 0.49 g of 2,2′-azobis(2-methylbutyronitrile) initiator. The polymerization starts quickly and reaches a maximum of exotherm. Once the maximum temperature is reached, the medium is placed at reflux for 2 hours.

A white precipitate is observed in the solvent (tert-butanol+2.5 wt % water). This white precipitate corresponds to the copolymer of the invention. The solvent is then removed using a rotary evaporator with oil heated to 90° C. and under a reduced pressure of 100 mbar. The resulting copolymer is then dried overnight at 70° C.

Copolymers A to E are manufactured according to the above protocol. The polymerizations are carried out at 15% by mass of active material in relation to the reaction medium. The amounts of the different monomers are adjusted to obtain the proportions shown in Table 2 below for each example.

TABLE 1
Composition of the Hydroalcoholic Solution

| Components | % (by mass) |
|---|---|
| Water | 28 |
| Polymer | 0.5 |
| Ethanol | 70 |
| Glycerin | 1.5 |

TABLE 2
Proportion of Monomers and Characteristics
in Water and in the Hydroalcoholic Solution

| | | | | Characteristics | | |
|---|---|---|---|---|---|---|
| | Monomers (mol %) | | | Viscosity in | Viscosity of the | Transmittance of |
| | ATBS | AA | TMPTA | water^{(a)} | hydroalcoholic gel^{(b)} | hydroalcoholic gel^{(c)} |
| A | 67.54 | 31.79 | 0.67 | 11600 | 4500 | 95 |
| B | 56.65 | 42.73 | 0.62 | 12200 | 5400 | 95 |
| C | 29.86 | 69.68 | 0.46 | 11300 | 6100 | 95 |
| D | 9.98 | 89.8 | 0.22 | 9200 | 5500 | 95 |
| E | 5.0 | 94.84 | 0.16 | 5700 | 5100 | 95 |
| F | 56.95 | 43 | 0.05* | 35000 | <1000 | <10 |
| G | 67.95 | 32 | 0.05* | 27000 | <1000 | <10 |
| H | 25.7 | 73.8 | 0.5* | 9500 | <1000 | 50 |
| I | 50.2 | 49.78 | 0.02* | 4000 | <1000 | 70 |
| J | 84.15 | 15 | 0.85 | 13200 | 3200 | 76 |
| K | 94.05 | 5 | 0.95 | 15500 | 4100 | 64 |

AA: acrylic acid
ATBS: 2-acrylamido-2-methylpropane sulfonic acid
TMPTA: trimethylolpropane triacrylate
^{(a)}Viscosity (cps) is measured at 1% copolymer weight in deionized water at 25° C., with a Brookfield RV module viscometer at 20 rpm.
^{(b)}Viscosity (cps) is measured at 0.5 wt % copolymer in hydroalcoholic solution at 25° C., with a Brookfield RV module viscometer at the speed of 20 rpm.
^{(c)}Transmittance is measured through a 1 cm thick sample of hydroalcoholic gel according to the formula in Table 1 for a wavelength of 420 nm.
*the cross-linking agent used is methylenebisacrylamide (MBA)

The viscosity and transmittance of Examples A to E according to the invention are fully satisfactory. Example E has an interesting viscosity and is transparent but has a gelatinous appearance.

Therefore, examples A to E, according to the invention, clearly show that the use of a polymer comprising the monomers at the concentrations indicated in Table 2, this polymer being obtained by precipitation polymerization in a solvent comprising at least one alcohol, makes it possible to obtain a transparent hydroalcoholic gel, which has a higher viscosity than the gels of the prior art, making the application of the gel to the skin simpler and more practical.

Counter-examples F and G are, respectively, examples 1a and 1b reproduced in an identical manner from patent EP 1 047 716.

Counter-examples H and I are, respectively, examples 1 and 2 reproduced in an identical manner from patent JP 09157130.

Counter-examples F through K do not make it possible to achieve sufficient viscosity. Moreover, the corresponding gels are partially opaque with low transmittance for counter-examples H to K or totally opaque with a transmittance of less than 10 for counter-examples F and G.

1-15. (canceled)

16. A use, for the manufacture of a hydroalcoholic composition comprising at least water and at least one alcohol, of a branched or cross-linked copolymer obtained from:
(i) 20 mol % to 94.99 mol % of at least one monomer containing an acrylic group and bearing at least one weak acid function,
(ii) 5 mol % to 79.99 mol % of at least one monomer bearing at least one strong acid function,
(iii) 0.01 mol % to 4 mol % of a cross-linking/branching agent selected from ethylene glycol diacrylate, polyethylene glycol dimethacrylate, diacrylamide, cyanomethylacrylate, vinyloxyethylacrylate, vinyloxymethacrylate, triallylamine, trimethylolpropane triacrylate, formaldehyde, glyoxal, glycidyl ethers, epoxies and mixtures thereof, said copolymer being obtained by precipitation polymerization, in a solvent comprising at least one alcohol.

17. A use, for thickening a hydroalcoholic composition comprising at least water and at least one alcohol, of a branched or cross-linked copolymer obtained from:
(i) 20 mol % to 94.99 mol % of at least one monomer containing an acrylic group and bearing at least one weak acid function,
(ii) 5 mol % to 79.99 mol % of at least one monomer bearing at least one strong acid function,
(iii) 0.01 mol % to 4 mol % of a cross-linking/branching agent selected from ethylene glycol diacrylate, polyethylene glycol dimethacrylate, diacrylamide, cyanomethylacrylate, vinyloxyethylacrylate, vinyloxymethacrylate, triallylamine, trimethylolpropane triacrylate, formaldehyde, glyoxal, glycidyl ethers, epoxies and mixtures thereof, said copolymer being obtained by precipitation polymerization, in a solvent comprising at least one alcohol.

18. The use according to claim 16, characterized in that the monomer containing an acrylic group and bearing at least one weak acid function is chosen from acrylic acid, methacrylic acid, itaconic acid, crotonic acid, fumaric acid, maleic acid, and mixtures thereof; said monomer being in acid form, partially salified, or fully salified.

19. The use according to claim 16, characterized in that 20% to 100 mol % of the strong acid functions are salified, more preferably 40% to 100 mol %, still more preferably 60 mol % to 100 mol %.

20. The use according to claim 16, characterized in that the monomer bearing at least one strong acid function is chosen from acrylamidoalkylsulphonic acids, such as 2-acrylamido-2-methylpropane sulphonic acid, said monomer being in acid form, partially salified or fully salified.

21. The use according to claim 16, characterized in that 10 mol % to 100 mol % of the weak acid functions are salified, preferably 20 mol % to 80 mol %, more preferably 30 mol % to 70 mol %.

22. The use according to claim 16, characterized in that the copolymer consists of the following monomers: at least one acrylic acid monomer and at least one 2-acrylamido-2-methylpropane sulfonic acid monomer.

23. The use according to claim 16, characterized in that the solvent consists of 90% to 100% by weight of an alcohol selected from methanol, ethanol, propan-1-ol, isopropanol, tert-butanol, and mixtures thereof and 0% to 10% by weight of water.

24. The use according to claim 16, characterized in that the branched or cross-linked copolymer is obtained from:
(i) 20 mol % to 89.99 mol % of at least one monomer containing an acrylic group and bearing at least one weak acid function,
(ii) 10 mol % to 79.99 mol % of at least one monomer bearing at least one strong acid function,
(iii) 0.01 mol % to 2 mol % of a cross-linking/branching agent.

25. A hydroalcoholic composition comprising:
a) 55% to 79.95% by weight of at least one alcohol A1,
b) 0.05% to 5% by weight of a branched or cross-linked copolymer obtained from:
(i) 20 mol % to 94.99 mol % of at least one monomer having an acrylic group and bearing at least one weak acid function,
(ii) 5 mol % to 79.99 mol % of at least one monomer bearing at least one strong acid function,
(iii) 0.01 mol % to 4 mol % of a cross-linking/branching agent selected from ethylene glycol diacrylate, polyethylene glycol dimethacrylate, diacrylamide, cyanomethylacrylate, vinyloxyethylacrylate, vinyloxymethacrylate, triallylamine, trimethylolpropane triacrylate, formaldehyde, glyoxal, glycidyl ethers, epoxies and mixtures thereof,
c) 15% to 44.95% by weight of water,
d) 0% to 10% by weight of at least one additive,
said copolymer being obtained by precipitation polymerization in a solvent comprising at least one alcohol.

26. The hydroalcoholic composition according to claim 25, characterized in that the alcohol A1 is chosen from methanol, ethanol, isopropanol, butanol, pentanol, hexanol, the isomers, and mixtures thereof.

27. The hydroalcoholic composition of claim 25, characterized in that the hydroalcoholic composition has a transmittance greater than 80% the transmittance corresponding to the percentage of light transmitted through a 1 cm thick sample in a quartz cell, at a wavelength between 400 nm and 800 nm.

28. The hydroalcoholic composition according to claim 25, characterized in that the additive comprises at least one moisturizing agent selected from allantoin; pyrrolidonecarboxylic acid and salts thereof; hyaluronic acid and salts thereof; sorbic acid and salts thereof; amino acids; polyhydroxy alcohols such as glycerin, propylene glycol, hexylene glycol, hexanetriol, ethoxydiglycol, dimethiconecopolyol, and sorbitol, as well as esters thereof; polyethylene glycol; glycolic acid and glycolate salts; chitosan; aloe vera extracts; algal extracts; honey and extracts thereof; inositol; lactic acid and lactate salts; D-panthenol; magnesium ascorbylphosphate; kojic acid; lactamide-monoethandamine; acetamide-monoethanolamine; their analogs, and mixtures thereof.

29. A hydroalcoholic composition according to claim 25, comprising:
a) 60% to 69.9% by weight of at least one alcohol,
b) 0.1% to 3% by weight of at least one branched or cross-linked copolymer according to claim 10,
c) 25% to 39.9% by weight of water.

30. The hydroalcoholic composition of claim 26, characterized in that the hydroalcoholic composition has a transmittance greater than 80% the transmittance corresponding to the percentage of light transmitted through a 1 cm thick sample in a quartz cell, at a wavelength between 400 nm and 800 nm.

31. The hydroalcoholic composition according to claim 26, characterized in that the additive comprises at least one moisturizing agent selected from allantoin; pyrrolidonecarboxylic acid and salts thereof; hyaluronic acid and salts thereof; sorbic acid and salts thereof; amino acids; polyhydroxy alcohols such as glycerin, propylene glycol, hexylene glycol, hexanetriol, ethoxydiglycol, dimethiconecopolyol, and sorbitol, as well as esters thereof; polyethylene glycol; glycolic acid and glycolate salts; chitosan; aloe vera extracts;
algal extracts; honey and extracts thereof; inositol; lactic acid and lactate salts; D-panthenol;
magnesium ascorbylphosphate; kojic acid; lactamide-monoethandamine; acetamide-monoethanolamine; their analogs, and mixtures thereof.

32. A hydroalcoholic composition according to claim 26, comprising:
a) 60% to 69.9% by weight of at least one alcohol,
b) 0.1% to 3% by weight of at least one branched or cross-linked copolymer according to claim 10,
c) 25% to 39.9% by weight of water.

33. The hydroalcoholic composition according to claim 27, characterized in that the additive comprises at least one moisturizing agent selected from allantoin; pyrrolidonecarboxylic acid and salts thereof; hyaluronic acid and salts thereof; sorbic acid and salts thereof; amino acids; polyhydroxy alcohols such as glycerin, propylene glycol, hexylene glycol, hexanetriol, ethoxydiglycol, dimethiconecopolyol, and sorbitol, as well as esters thereof; polyethylene glycol; glycolic acid and glycolate salts; chitosan; aloe vera extracts; algal extracts; honey and extracts thereof; inositol; lactic acid and lactate salts; D-panthenol; magnesium ascorbylphosphate; kojic acid; lactamide-monoethandamine; acetamide-monoethanolamine; their analogs, and mixtures thereof.

34. A hydroalcoholic composition according to claim 27, comprising:
a) 60% to 69.9% by weight of at least one alcohol,
b) 0.1% to 3% by weight of at least one branched or cross-linked copolymer according to claim 10,
c) 25% to 39.9% by weight of water.

35. A hydroalcoholic composition according to claim 33, comprising:
a) 60% to 69.9% by weight of at least one alcohol,
b) 0.1% to 3% by weight of at least one branched or cross-linked copolymer according to claim 10,
c) 25% to 39.9% by weight of water.
